(19) United States
(12) Patent Application Publication
(10) Pub. No.: US 2019/0228955 A1
(43) Pub. Date: Jul. 25, 2019

(54) METHODS OF DETECTING CHEMICALS

(71) Applicant: Purdue Research Foundation, West Lafayette, IN (US)

(72) Inventors: Paul G. Wenthold, West Lafayette, IN (US); Sabyasachy Mistry, West Lafayette, IN (US)

(73) Assignee: Purdue Research Foundation, West Lafayette, IN (US)

(21) Appl. No.: 16/252,676

(22) Filed: Jan. 20, 2019

Related U.S. Application Data
(60) Provisional application No. 62/620,066, filed on Jan. 22, 2018.

(51) Int. Cl. H01J 49/00; H01J 49/16; G01N 33/02

(52) U.S. Cl. CPC H01J 49/0031; G01N 33/02; H01J 49/165

(57) ABSTRACT

A method of identifying phenolic compounds in a compound under test (CUT) is disclosed. The method includes mixing the CUT with a buffer solution to generate a buffered compound, mixing the buffered compound with a Gibbs reagent and allowing reaction for a predetermined amount of time to generate an indophenol; inputting the indophenol to a mass spectrometer, generating spectra of the indophenol, and analyzing the spectra to determine presence of phenolic compounds in the indophenols.

### CROSS-REFERENCE TO RELATED APPLICATIONS

The present patent application is related to and claims the priority benefit of U.S. Provisional Patent Application Ser. No. 62/620,066 filed Jan. 22, 2018, the content of which is hereby incorporated by reference in its entirety into the present disclosure.

### STATEMENT REGARDING GOVERNMENT FUNDING

This invention was made with government support under CHE15-65755 awarded by the National Science Foundation. The government has certain rights in the invention.

### TECHNICAL FIELD

The present disclosure generally relates to a method of detecting and measuring chemical contents, and in particular, to a method of detecting phenol-based compounds using mass spectrometry.

### BACKGROUND

This section introduces aspects that may help facilitate a better understanding of the disclosure. Accordingly, these statements are to be read in this light and are not to be understood as admissions about what is or is not prior art.

Phenol-based compounds are found all around us. They can be seen in naturally-occurring materials as well as man-made materials. In the naturally-occurring category, examples include biological molecules, such as tyrosine and L-dopa, food components, such as capsaicin or red wine tannins, and even drugs, such as morphine and tetrahydrocannabinol. In the man-made category, examples include industrial waste which can have phenol concentrations as high as 10 g/L. For a variety of reasons, e.g., food and drug safety, it is of import to identify composition of material, and in particular presence and concentration of phenolic compounds.

There have been many methods developed to detect phenols. One such method is colorimetric detection of phenols. Such methods can be traced back almost 200 years, to the detection of orcinol. For example, Liebermann in late 1800's showed that phenols, including phenol, orcinol and thymol, can be transformed into dark-blue-colored indophenol dyes by reaction with sulfuric acid and ammonia as shown below.

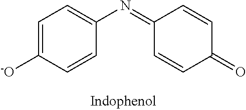

Then in 1927, Gibbs reported a convenient reagent for the detection of phenols by converting them to indophenols. The Gibbs reaction (eq 1), utilizing the Gibbs Reagent, 2,6-dichloro- or dibromo-4-(chloroimino)cyclohexa-2,5-dien-1-one, has since been a standard approach for the detection of phenols.

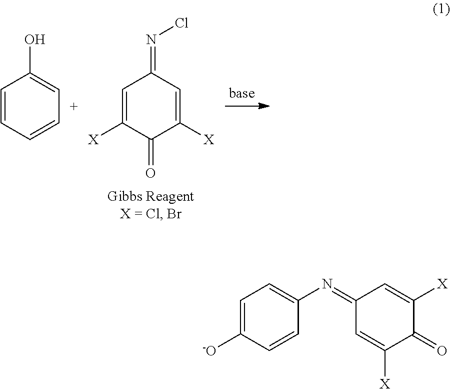

One of the key features that makes the Gibbs procedure a useful approach for the detection of phenols is the robust color of the indophenol product, which allows for easy spectrophometric detection. In recent years, applications of the Gibbs procedure have been reported for the detection of phenol-based pharmaceuticals, capsaicin, and for monitoring the presence of phenol. In all of these applications, the indophenol is detected spectrophometrically.

However, one of the limitations in spectrophometric detection methodology of the Gibbs approach for detection of phenols is that the absorption peak is not very sensitive to substitution of the phenol. Consequently, while the standard Gibbs method is useful for determining total phenol content, it is generally not capable of distinguishing between individual substituted phenols. To address this limitations, several other approaches have been developed.

To address the above-stated limitation, a separation step is needed for the detection of specific phenol derivatives. Other non-colorometric methods have been utilized in the prior art to detect phenols in compounds. One such method has been established by Lowe et al. who have examined and detected the Gibbs reaction using electrochemistry and cyclic voltammetry, and have shown that it can be used to detect, various compounds, e.g., tetrahydrocannabinol (THC).

According to yet another non-colorometric method, to further selectively separate phenol compounds, liquid chromatography or gas chromatography methods have also been used to establish presence of different phenol substitutions.

According to still yet another non-colorometric method, Josephy and Lenkinski have characterized the Gibbs product formed from tert-butylhydroxyanisole (BHA) by using electron ionization mass spectrometry based on electron-ionization spectra for some protonated indophenols (neutral phenols) which are generally available.

All the above methods, however, suffer from lack of sensitivity, lack of selectivity, or lack of ability to identify position of the substituent (i.e., ortho, meta, or para) on the phenol ring.

Therefore, there is an unmet need for a novel approach that can identify concentration of phenols in a compound, simultaneously and selectively detect multiple phenol derivatives without requiring prior separation, and further provide position of the substituent on the phenol ring.

### SUMMARY

A method of identifying phenolic compounds in a compound under test (CUT) is disclosed. The method includes mixing the CUT with a buffer solution to generate a buffered compound. The method also includes mixing the buffered compound with a Gibbs reagent and allowing reaction for a predetermined amount of time to generate an indophenol. In addition, the method includes inputting the indophenol to a mass spectrometer. Furthermore, the method includes generating spectra of the indophenol, and analyzing the spectra to determine presence of phenolic compounds in the indophenols.

A method of identifying isomeric phenolic compounds in a compound under test (CUT), is also disclosed. The method includes mixing the CUT with a buffer solution to generate a buffered compound. The method also includes mixing the buffered compound with a Gibbs reagent and allowing reaction for a predetermined amount of time to generate an indophenol. Furthermore, the method includes inputting the indophenol to a mass spectrometer, and generating spectra of the indophenol. The method also includes disassociating ions utilizing a collision induced disassociation (CID) stage, and analyzing the spectra to determine i) presence of phenolic compounds in the indophenols, and ii) identify isomers of the phenolic compounds.

A method of quantifying phenolic concentration of compounds in a compound under test, is also disclosed. The method include mixing the CUT with a buffer solution to generate a buffered compound, mixing the buffered compound with a Gibbs reagent and allowing reaction for a first predetermined amount of time to generate a first set of one or more indophenols, inputting the first set of one or more indophenols to a mass spectrometer, generating spectra of the first set of one or more indophenols, identifying a first set of one or more peaks associated with the first set of one or more indophenols, identifying a phenolic compound to be used as an internal standard with a peak for a corresponding indophenol of the internal standard at an m/z having a separation from the first set of one or more peaks of at least 5, mixing the first set of one or more indophenols with the internal standard having a predetermined concentration for a second predetermined amount of time to generate a second set of one or more indophenols, inputting the second set of one or more indophenols to the mass spectrometer, generating spectra of the second set of one or more indophenols, generating calibration curves for each of the first set of one or more indophenols, wherein the calibration curve represents a ratio of intensity of each of the first set of one or more indophenols to intensity of the internal standard vs. concentration of each of the first set of one or more indophenols, and obtaining the concentration of each of the first set of indophenols.

### BRIEF DESCRIPTION OF DRAWINGS

FIG. 1a is a mass spectrum of the Gibbs reagent.

FIG. 1b is a mass spectrum of a phenol.

FIG. 1c is a mass spectrum of an indophenol of the phenol and the Gibb reagent.

FIG. 1d is a deconvoluted version of the mass spectrum of FIG. 1c.

FIG. 2 is mass spectra for the indophenol of the ortho-cresol (R═CH₃) after mixing with the Gibbs reagent (the top panel represents the full spectrum of the indophenol while the bottom panel represents the deconvoluted spectrum).

FIG. 3 is mass spectra for the indophenol of the meta-cresol (R═CH₃) after mixing with the Gibbs reagent (the top panel represents the full spectrum of the indophenol while the bottom panel represents the deconvoluted spectrum).

FIG. 4 is mass spectra for the indophenol of the ortho-hydroxyanisole (R═OCH₃) after mixing with the Gibbs reagent (the top panel represents the full spectrum of the indophenol while the bottom panel represents the deconvoluted spectrum).

FIG. 5 is mass spectra for the indophenol of the meta-hydroxyanisole (R═OCH₃) after mixing with the Gibbs reagent (the top panel represents the full spectrum of the indophenol while the bottom panel represents the deconvoluted spectrum).

FIG. 6 is mass spectra for the indophenol of the catechol (R═OH) after mixing with the Gibbs reagent (the top panel represents the full spectrum of the indophenol while the bottom panel represents the deconvoluted spectrum).

FIG. 7 is mass spectra for the indophenol of the resorcinol (R═OH) after mixing with the Gibbs reagent (the top panel represents the full spectrum of the indophenol while the bottom panel represents the deconvoluted spectrum).

FIG. 8 is mass spectra for the indophenol of the ortho-aminophenol (R═NH₂) after mixing with the Gibbs reagent (the top panel represents the full spectrum of the indophenol while the bottom panel represents the deconvoluted spectrum).

FIG. 9 is mass spectra for the indophenol of the meta-aminophenol (R═NH₂) after mixing with the Gibbs reagent (the top panel represents the full spectrum of the indophenol while the bottom panel represents the deconvoluted spectrum).

FIG. 10 is mass spectra for 1-naphthol and the corresponding Gibbs reagent indophenol (the top panel represents the full spectrum of the indophenol while the bottom panel represents the deconvoluted spectrum).

FIG. 11 is mass spectra for tetrahydro-1-napthol and the corresponding Gibbs reagent indophenol (the top panel represents the full spectrum of the indophenol while the bottom panel represents the deconvoluted spectrum).

FIGS. 12a and 12b are collision induced disassociation (CID) spectra of the indophenols formed from ortho-(12a) and meta-cresol (12b), respectively.

FIGS. 12c and 12d are CID spectra of the indophenols of catechol (R═OH) or resorcinol (R═OH), respectively.

FIGS. 12e and 12f are CID spectra of the indophenols of ortho-aminophenol (R═NH₂) and meta-aminophenol (R═NH₂), respectively.

FIGS. 12g and 12h are CID spectra of the indophenols of guaiacol and meta-hydroxyanisole (R═OCH₃), respectively.

FIG. 13 is a flowchart according to the present disclosure of a process by which substituents in a compound under test are identified using the Gibbs reagent and mass spectrometry.

FIGS. 14a and 14b are deconvoluted mass spectra of Gibbs products obtained from a 1:1 mixture of phenol and o-cresol taken at 5 min of mixing (FIGS. 14a) and 30 min of mixing (FIG. 14b).

FIG. 15 is a calibration graph of intensity vs. concentration of phenols in μmol/L with o-cresol used as an internal standard.

FIG. 16 is a flow chart according to the present disclosure of a process for quantifying concentrations of phenolic compounds in a compound under test utilizing an internal standard.

FIGS. 17a, 17b, 17c, and 17d are deconvoluted mass spectra for various liquid smoke (hickory (17a), mesquite (17b), pecan (17c), and apple (17d)).

FIG. 18 is a calibration graph of intensity vs. concentration of catechol in mg/L with 5,6,7,8-tetrahydro-1-napthol (THN) used as an internal standard.

FIG. 19 is a calibration graph of intensity vs. concentration of guaiacol in mg/L with 5,6,7,8-tetrahydro-1-napthol (THN) used as an internal standard.

FIG. 20 is a calibration graph of intensity vs. concentration of syringol in mg/L with 5,6,7,8-tetrahydro-1-napthol (THN) used as an internal standard.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

A novel approach is disclosed herein that can identify concentration of phenols in a compound, simultaneously and selectively detect multiple phenol derivatives without requiring prior separation, and further provide position of the substituent on the phenol ring.

Accordingly, the present disclosure describes detection of Gibbs products by using electrospray-ionization mass spectrometry (ESI-MS). This approach is contrary to accepted wisdom because use of the Gibbs reagent for detection of phenols has been since the early days of the Gibbs reagent been limited to colorimetric approaches. However, the inventors found that use of mass spectrometery surprisingly provides robust selectivity in identifying phenolic compounds based on the Gibbs reagent and the indophenols resulting therefrom. Since indophenols are naturally anionic, they are readily detected by ESI-MS. An important advantage of using mass spectrometry for the detection of indophenols is that it readily distinguishes between different substituents when in the ortho- and meta-positions. In the present disclosure, ESI mass spectra for indophenols obtained in the Gibbs reaction of simple substituted phenols are provided, which show a robust application for simultaneous detection of components in a mixture, including identification and quantification.

Accordingly, the present disclosure provides a novel method for detecting phenols of unknown concentration in a compound, by reacting the compound with the Gibbs Reagent to form indophenols, followed by mass spectrometric detection. Unlike the standard Gibbs reaction, which uses a colorometric approach, the use of mass spectrometry allows for simultaneous detection of differently substituted phenols.

Use mass spectrometry to detect the indophenol product allows for a more specific determination of phenol composition. The relative yields of Gibbs products additionally allows for quantification of phenol components, by measuring intensity against an internal standard. Mass spectrometric analysis also allows for investigation of the product structures, particularly for the reactions with para-substituted phenols, which can react either by direct substitution of the substituent, or at another site within the phenol, preserving the para substituent. The method is shown to work for the analysis of liquid smoke, and is therefore amenable for the analysis of wood-smoke condensates

The procedure is demonstrated to work for a large variety of phenols without para-substitution. With para-substituted phenols, Gibbs products are still often observed, but the specific product depends on the substituent. For para groups with high electronegativity, such as methoxy or halogens, the reaction proceeds by displacement of the substituent. For groups with lower electronegativity, such as amino or alkyl groups, Gibbs products are observed that retain the substituent, indicating that the reaction occurs at the ortho or meta positions. In mixtures of phenols, the relative intensities of the Gibbs products are proportional to the relative concentrations, and concentrations as low as 1 μmol/L can be detected. In one example, the novel method of the present disclosure is thus applied to a qualitative analysis of commercial liquid smoke, and it is found that hickory, mesquite, pecan, and apple flavors represent significantly different phenolic composition.

Exemplary aspects of the novel methodology of the present disclosure is now provided.

### Sample Preparation (General Procedure)

Samples were prepared by mixing an exemplary volume (e.g., 5 mL) of a solution of the Gibbs Reagent in methanol (e.g., 60 mmol/L) with 10 mL of an aqueous potassium phosphate dibasic solution (deionized water, 40 mmol/L, pH 9.5) containing substrate. After mixing, the solutions were stirred at room temperature for the allotted time, after which 50 μL of the solution was diluted to 4 mL in water-methanol solution (1:1), for ESI-MS analysis. Solutions for individual samples contained 0.25 mmol of substrate in the phosphate buffer. Spectra are measured 5 minutes after mixing—unless otherwise noted.

For the mixture of phenol and o-cresol, 10 mL of the initial solution containing o-cresol and phenol were mixed with 5 mL methanol solution of the Gibbs Reagent (100 mmol/L). The reaction mixtures was stirred for 5 minutes and 30 minutes, respectively, before diluting with 1:1 methanol as described above, followed by mass spectrometric analysis. The concentration of o-cresol in the initial solution was chosen so to obtain a concentration of 3 μmol/L in the final electrosprayed solution. Similarly, the concentration of phenol in the initial solution was chosen so to obtain concentrations of 1, 2, 5, 10, 25 and 50 μmol/L in the final solution.

### ESI-MS

Electrospray ionization mass spectra were obtained on a commercial LCQ-DECA (THERMO ELECTRON CORPORATION, San Jose, Calif., USA) quadrupole ion trap mass spectrometer, equipped with an ESI source, operating in negative ion mode. Substrate solutions in a methanol: water mixture (1:1) were introduced into the source directly at a flow rate of 10 μL/min. Electrospray and ion focusing conditions were varied to maximize the signal of the ion of interest.

### Sample Preparation (Liquid Smoke)

K₂HPO₄ (0.5 mmol, 87 mg) was added directly to 5 mL of the corresponding commercial smoke sample directly and mixed with 10 mL methanol solution of the Gibbs Reagent (0.5 mmol, 105 mg). The reaction mixture was stirred at room temperature for 5 minutes. 200 μL of the reaction mixture was dissolved in 4 mL water-methanol (1:1) solution for spectrometric analysis.

### Spectral Analysis

An important advantage of using the Gibbs Reagent for derivatization is that the chlorine atoms make products containing the Gibbs Reagent readily detectable by the isotopic pattern. Therefore, the spectra are deconvoluted using the isotope pattern for ions containing two chlorine atoms (1:0.648:0.105, where 1 represents the first peak, 0.648 represents the second peak and the 0.105 represents the third peak) to eliminate the peaks that cannot contain the Gibbs Reagent. Thus, the deconvolution process acts as a filter to eliminate peaks that do not have the double chlorine ions (i.e., peaks associated with non-indophenols). It is this ability to specifically detect phenoxides and eliminate non-Gibbs products that makes the Gibbs approach preferable to the spectrometric analysis of the mixture using ESI-MS.

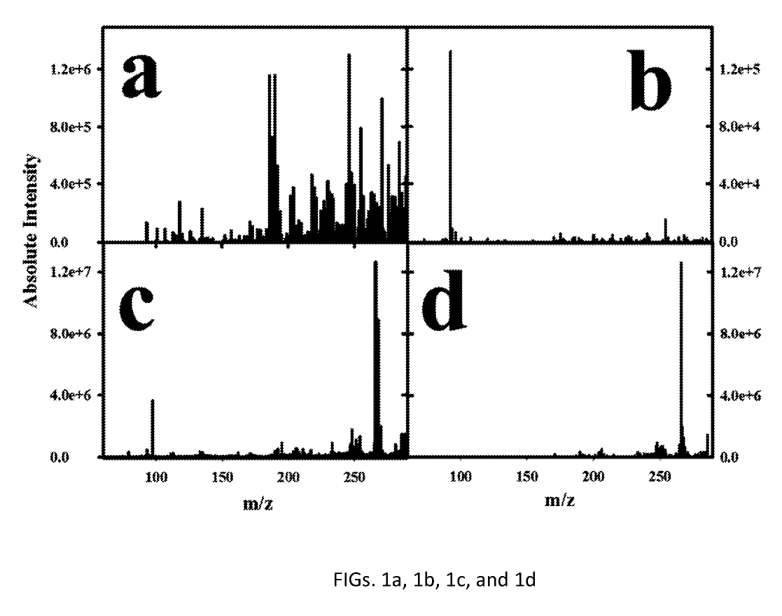

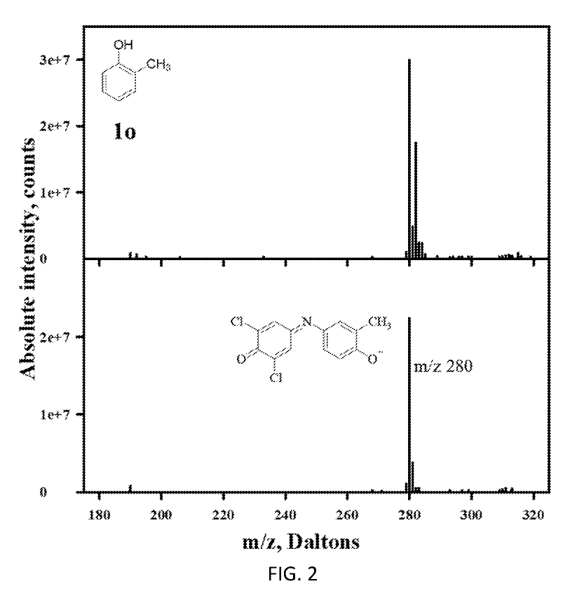

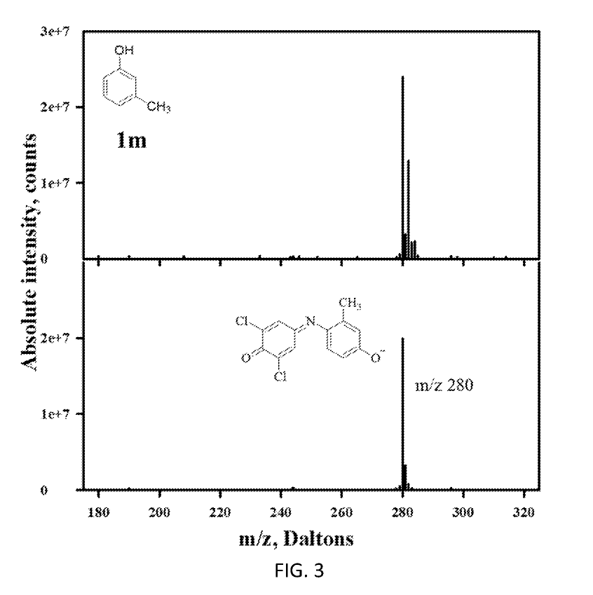

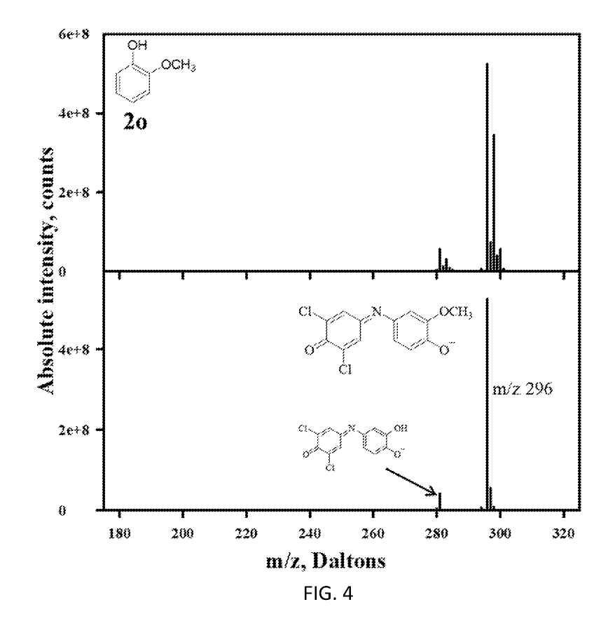

Referring to FIGS. 1a, 1b, and 1c, spectra obtained from the ESI-MS of the Gibbs reagent alone in buffer (FIG. 1a), phenol alone in buffer (FIG. 1b), phenol and the Gibbs reagent in buffer 5 minutes after mixing (FIG. 1c) are provided. As shown in FIG. 1a, in the absence of phenol, the MS of the Gibbs reagent is non-descript, and shows a large number of unidentifiable ionic products. FIG. 1b provides a clear indication of phenoxide (m/z 93). However, in the presence of other acidic substrates, the phenoxide product would be expected to be a minor product. FIG. 1c shows the spectrum obtained 5 minutes after mixing Gibbs reagent and phenol in the buffer solution. The spectrum is dominated by the Gibbs indophenol product, as indicated by the characteristic isotopic pattern. Moreover, despite having phenol at the same concentration as in FIG. 1b, the absolute intensity increases by a factor of about 100 for the detection of the Gibbs product, not considering the fact that signal is distributed over multiple isotope peaks. Therefore, the total signal of the phenol derivative in FIG. 1c is nearly 200 times greater than that of phenol in FIG. 1b. For example, in FIG. 1c, the intensity is 1.2×10⁵ for the phenoxide. However, the intensity of the indophenol is about 1.2×10⁷, evidencing the robust ionic nature of the indophenols, making mass spectrometry a viable choice for detection of phenols using the Gibbs reagent. FIG. 1d provides the spectrum obtained after deconvolution, to account for the isotope peaks. Although the initial spectrum was originally very clean, the deconvoluted spectrum is even more so, with more than 85% of the signal attributable to indophenol. The deconvolution process is a filtering process based on the two chlorine ions in the indophenols, further described below.

Gibbs products are also readily detected by ESI-MS for other substituted phenols, included ortho- and meta-cresol (1o and 1m), ortho- and meta-hydroxyanisole (2o and 2m), catechol (3o), resorcinol (3m), 2-aminophenol (4), 1-naphthol (5), and tetrahydro-1-napthol (6) as provided below:

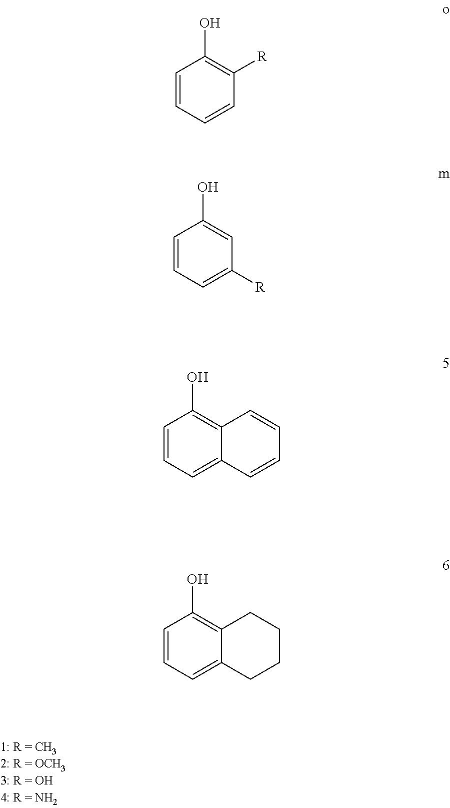

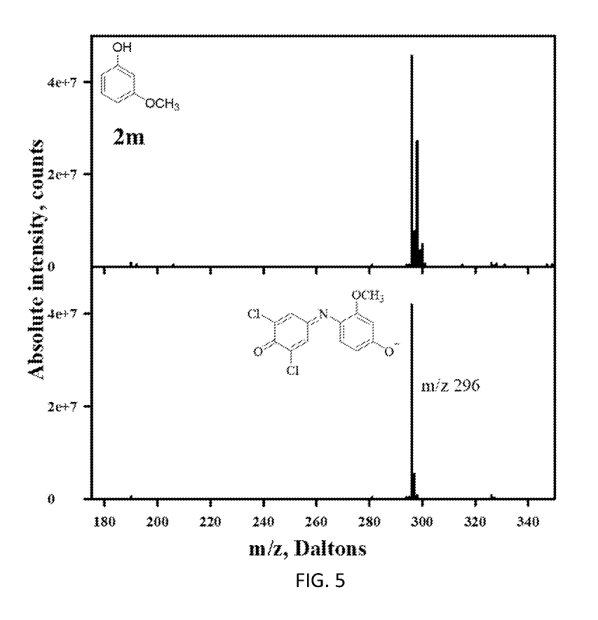

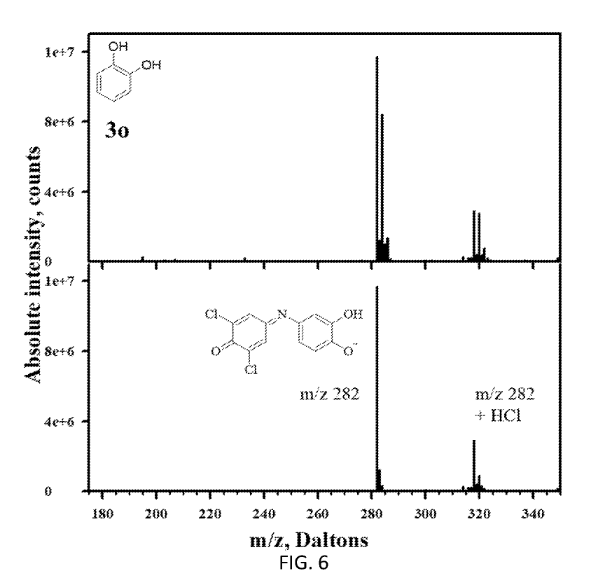

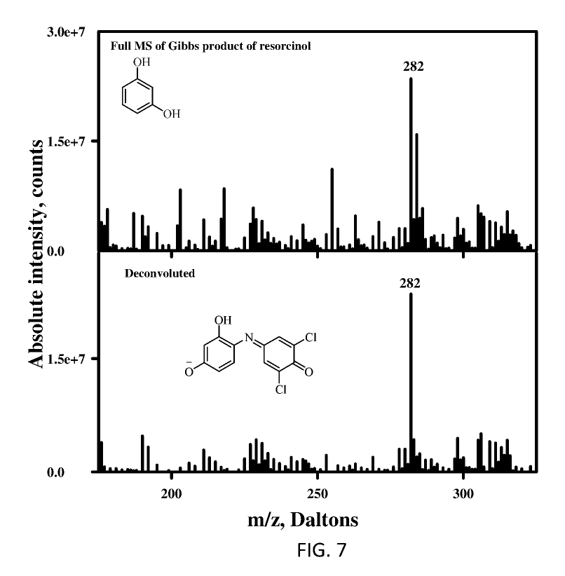

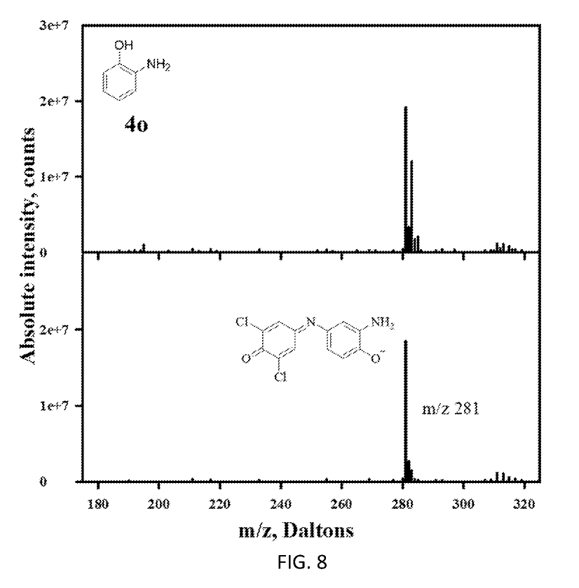

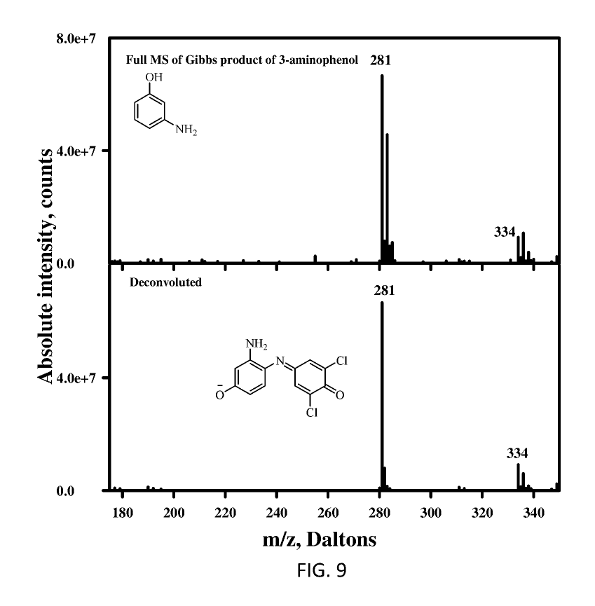

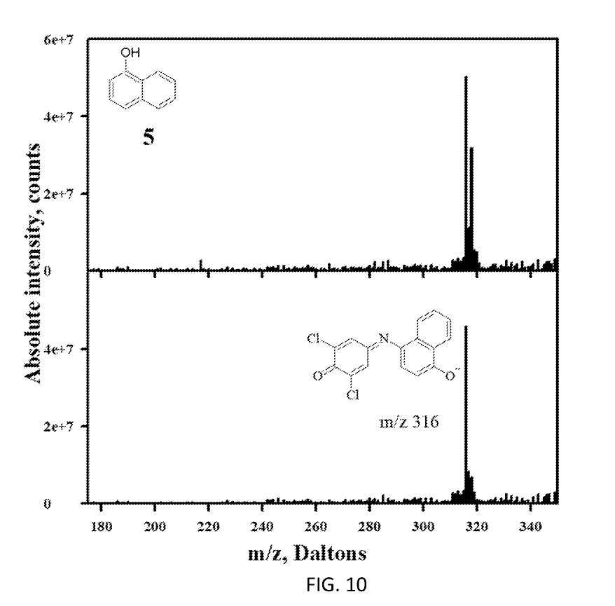

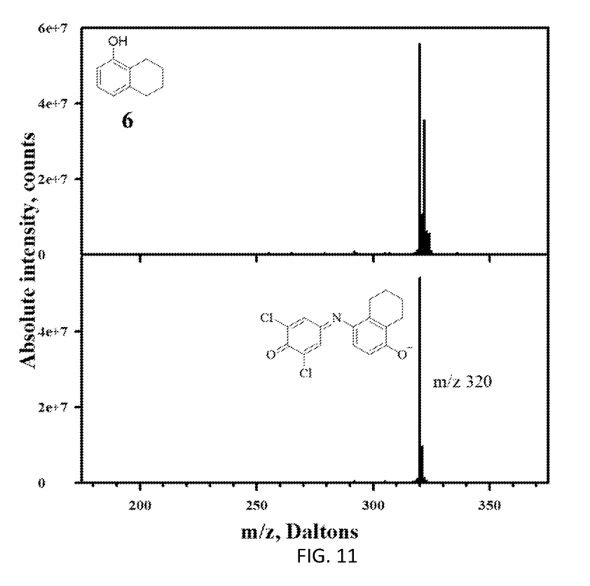

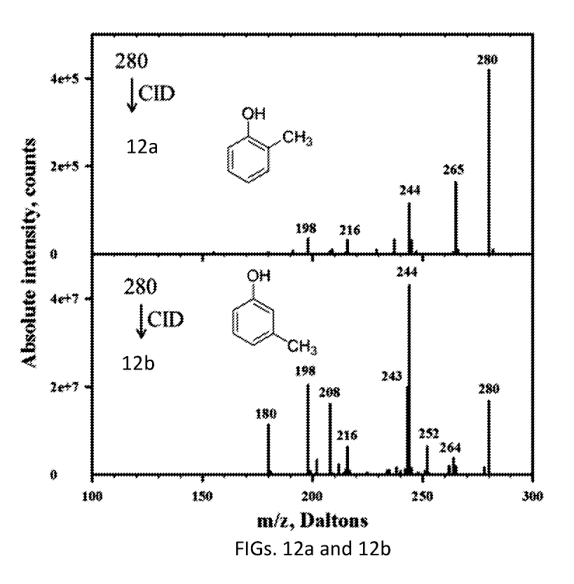

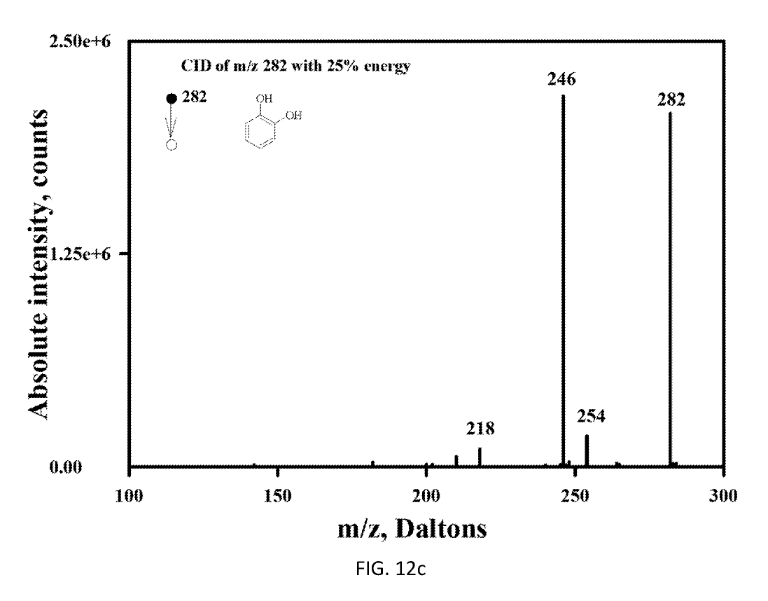

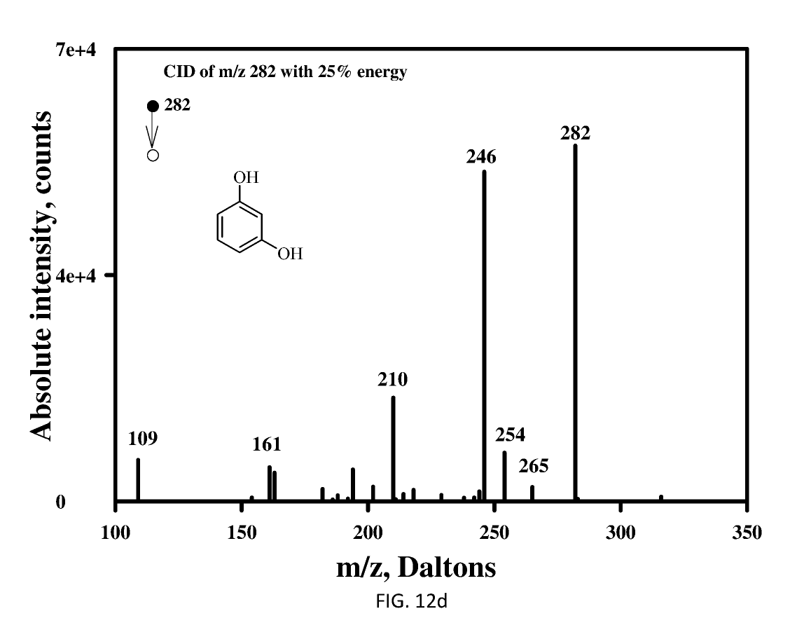

Spectra for each of these constituents at the ortho and meta positions in the indophenols resulting from mixing with the Gibbs reagent for those positions are shown in FIGS. 2-8. For example, FIG. 2 shows the spectra for the indophenol of the ortho-cresol (R═CH₃) after mixing with the Gibbs reagent. The top panel of FIG. 2 represents the full spectrum of the indophenol while the bottom panel represents the deconvoluted spectrum. FIG. 3 shows the spectra for the indophenol of the meta-cresol (R═CH₃) after mixing with the Gibbs reagent. The top panel of FIG. 3 represents the full spectrum of the indophenol while the bottom panel represents the deconvoluted spectrum. A comparison of FIGS. 2 and 3 reveals a peak at m/z of 280 which represents the indophenol of cresol (R═CH₃) regardless of the ortho-or meta-positions. FIG. 4 shows the spectra for the indophenol of the ortho-hydroxyanisole (R═OCH₃) after mixing with the Gibbs reagent. The top panel of FIG. 4 represents the full spectrum of the indophenol while the bottom panel represents the deconvoluted spectrum. FIG. 5 shows the spectra for the indophenol of the meta-hydroxyanisole (R═OCH₃) after mixing with the Gibbs reagent. The top panel of FIG. 5 represents the full spectrum of the indophenol while the bottom panel represents the deconvoluted spectrum. A comparison of FIGS. 4 and 5 reveals a peak at m/z of 296 which represents the indophenol of hydroxyanisole (R═OCH₃) regardless of the ortho or meta positions. FIG. 6 shows the spectra for the indophenol of the catechol (R═OH) after mixing with the Gibbs reagent. The top panel of FIG. 6 represents the full spectrum of the indophenol while the bottom panel represents the deconvoluted spectrum. FIG. 7 shows the spectra for the indophenol of the resorcinol (R═OH) after mixing with the Gibbs reagent. The top panel of FIG. 7 represents the full spectrum of the indophenol while the bottom panel represents the deconvoluted spectrum. A comparison of FIGS. 6 and 7 reveals a peak at m/z of 282 which represents the indophenol of catechol or resorcinol (R═OH), respectively. FIG. 8 shows the spectra for the indophenol of the ortho-aminophenol (R═NH₂) after mixing with the Gibbs reagent. The top panel of FIG. 8 represents the full spectrum of the indophenol while the bottom panel represents the deconvoluted spectrum. FIG. 9 shows the spectra for the indophenol of the meta-aminophenol (R═NH₂) after mixing with the Gibbs reagent. The top panel of FIG. 9 represents the full spectrum of the indophenol while the bottom panel represents the deconvoluted spectrum. A comparison of FIGS. 8 and 9 reveals a peak at m/z of 281 represents the indophenol of aminophenol (R═NH₂) regardless of the ortho or meta positions. FIG. 10 shows the spectra for 1-naphthol and the corresponding Gibbs reagent indophenol. The top panel of FIG. 10 represents the full spectrum of the indophenol while the bottom panel represents the deconvoluted spectrum. A closer look at FIG. 10 reveals a peak at m/z of 316 which represents the indophenol of 1-naphthol. FIG. 11 shows the spectra for tetrahydro-1-napthol and the corresponding Gibbs reagent indophenol. The top panel of FIG. 11 represents the full spectrum of the indophenol while the bottom panel represents the deconvoluted spectrum. A closer look at FIG. 11 reveals a peak at m/z of 320 which represents the indophenol of tetrahydro-1-napthol.

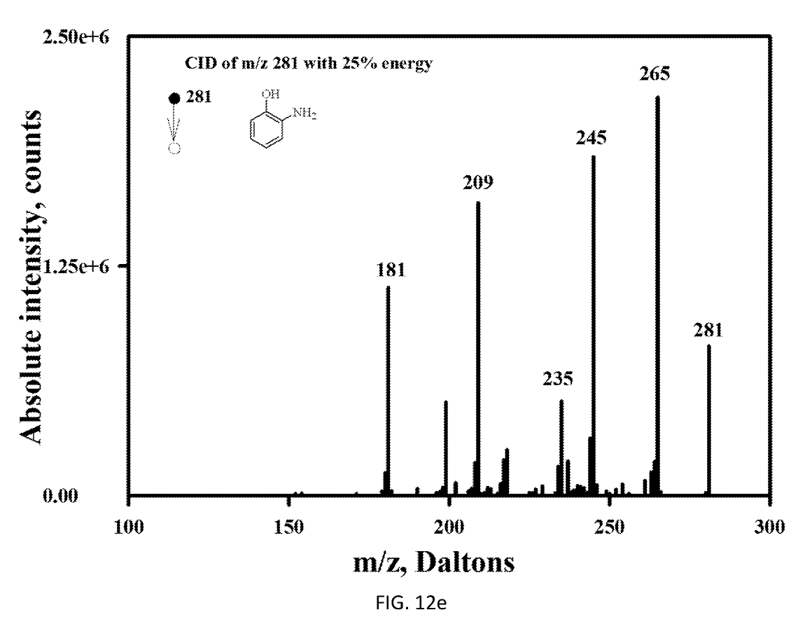

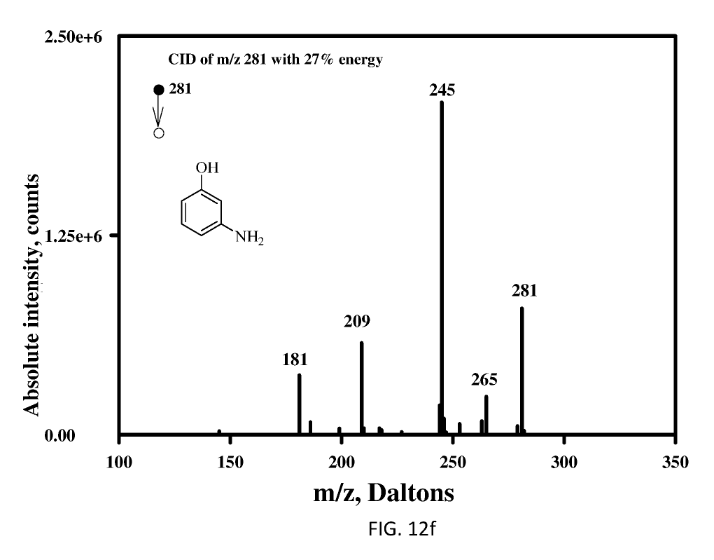

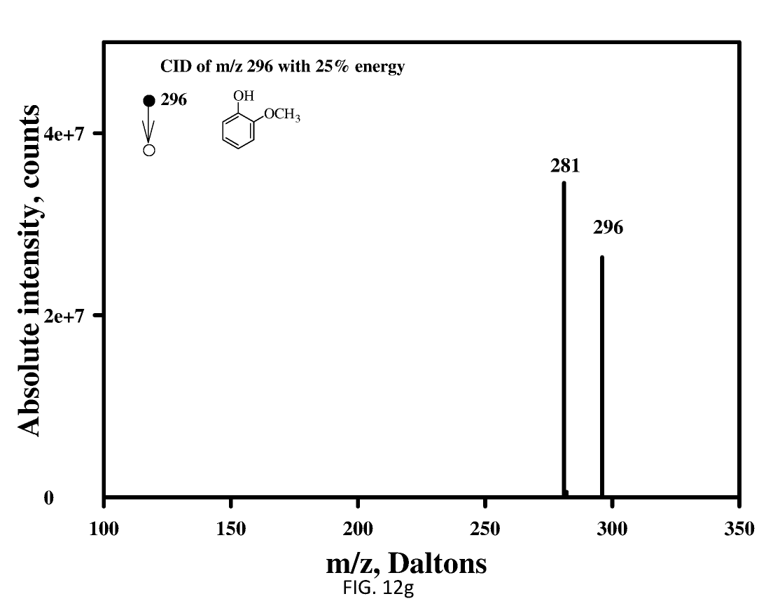

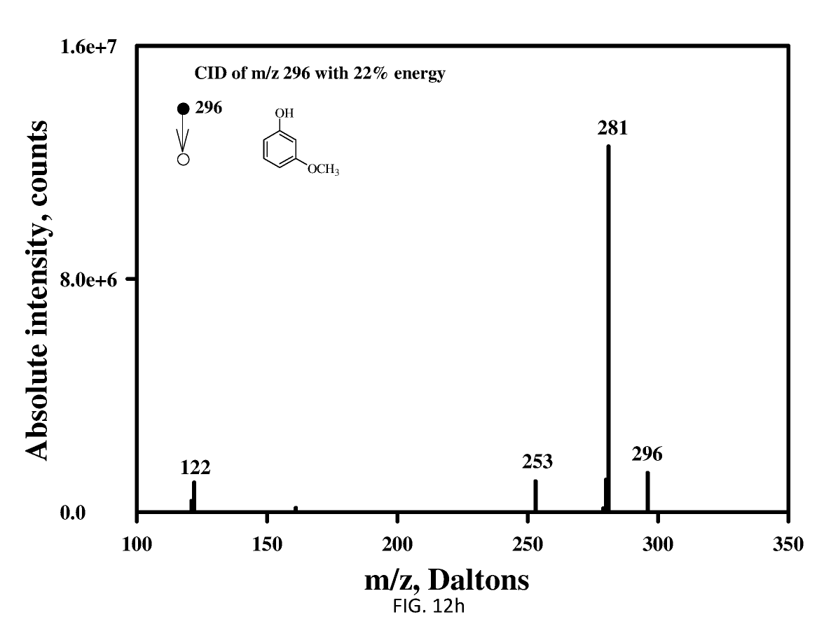

While mass spectrometry provides a robust approach for detection of phenol when mixed with the Gibbs reagent, thus generating indophenols as shown in eq. 1, mass spectrometry by itself is not well suited to distinguish between isomers of phenols. This limitation was already observed in the ortho and meta positions of the same substituents (see pairs of FIGS. 2 and 3, 4 and 5, 5 and 7, and 8 and 9). Thus in order to distinguish between these isomers (which have the same mass to charge ratio but have substituents at different positions), an additional step is needed. The extra step includes a collision induced disassociation (CID) stage, known to a person having ordinary skill in the art (e.g., U.S. Pat. No. 6,590,203 to Kato, contents of which is hereby incorporated by reference into the present disclosure in its entirety), and the associated CID spectrum of fragments. To demonstrate this added step, reference is now made to FIGS. 12a and 12b which show the CID spectra of the indophenols formed from ortho-(12a) and meta-cresol (12b), respectively. Significant differences are observed between the spectra, with methyl loss being a major dissociation pathway for the ortho-isomer, but not observed for meta, whereas m/z 252, 208, and 180 are exclusively observed for the meta-isomer. Therefore, in reference to FIG. 12a, for an indophenol of cresol (R═CH₃) at the ortho position, the CID spectrum represents peaks at m/z of 198, 216, 244, 265, and 280, while in reference to FIG. 12b, for an indophenol of cresol (R═CH₃) at the meta position, the CID spectrum represents peaks at m/z of 180, 198, 208, 216, 243, 244, 252, 264, and 280. Further reference is made to FIGS. 12c and 12d which show the CID spectra of the indophenols of catechol (R═OH)or resorcinol (R═OH), respectively. Therefore, in reference to FIG. 12c, for the indophenol of catechol (R═OH), the CID spectrum represents peaks at m/z of 218, 246, 254, and 282, while in reference to FIG. 12d, for the indophenol of resorcinol (R═OH), the CID spectrum represents peaks at m/z of 109, 161, 210, 246, 254, 265, and 282. Further reference is made to FIGS. 12e and 12f which show the CID spectra of the indophenols of ortho-aminophenol (R═NH₂) and meta-aminophenol (R═NH₂), respectively. Therefore, in reference to FIG. 12e, for the indophenol of ortho-aminophenol (R═NH₂), the CID spectrum represents peaks at m/z of 181, 199, 209, 235, 245, 265, and 281, while in reference to FIG. 12f, for the indophenol of meta-aminophenol (R═NH₂), the CID spectrum represents peaks at m/z of 181, 209, 245, 265, and 281. Further reference is made to FIGS. 12g and 12h which show the CID spectra of the indophenols of guaiacol, the CID spectrum represents peaks at m/z of 281 and 296 (FIG. 12g) and meta-hydroxyanisole (R═OCH₃), the CID spectrum represents peaks at m/z of 122, 253, 281, and 296. Although not all isomeric pairs show such dramatic differences, FIGS. 12a, 12b, 12c, 12d, 12e, 12f, and 12g show that CID of the indophenols can be used to distinguish between isomeric phenols.

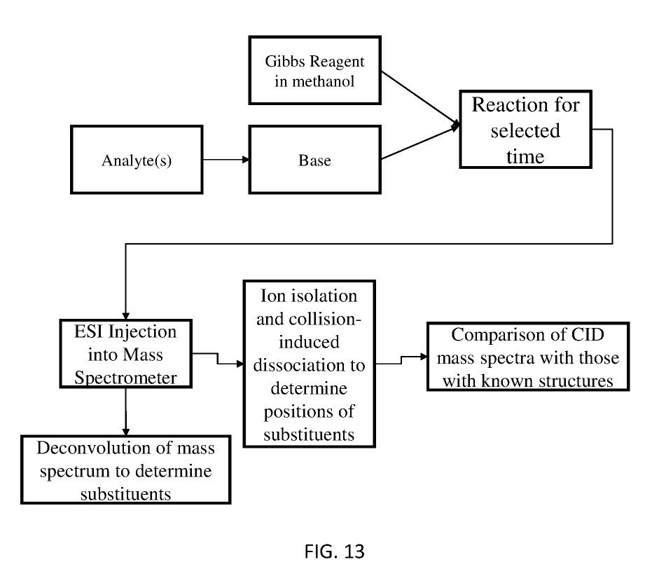

Therefore, according to one embodiment of the present disclosure, initially a mass spectrum of a phenolic Gibbs product (i.e., an indophenol as in eq. 1) is obtained. Reviewing the spectrum, phenolic compounds based on different substituents can be identified based on the m/z numbers (see FIGS. 2-11). In order to clean up the spectra from unwanted peaks, the spectra can be deconvolved to remove peaks lacking the two chlorine ions of the indophenols. Once the phenolic compounds have been identified, then isomers (i.e., identification of positions of substituents) can be identified by subsequently or simultaneously using the CID spectra. Referring to FIG. 13, a flowchart is provided showing the steps described above.

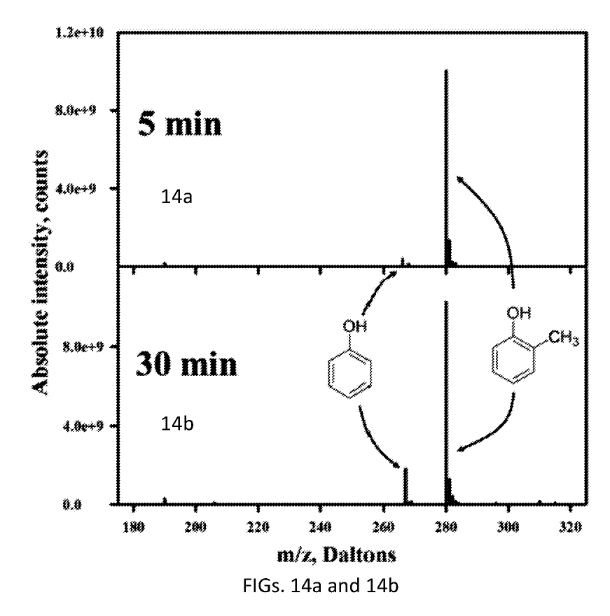

The next topic addressed in the present disclosure is sensitivity. As discussed above, an advantages of using mass spectrometry to detect the indophenol products of the Gibbs reaction is that it can be used for simultaneous detection of multiple phenol derivatives without separation. This is illustrated by the spectrum of 1:1 mixture of phenol and o-cresol (lo), each at 0.25 mmol/L concentration, shown in FIGS. 14a and 14b which are spectra of indophenols of cresol (R═CH₃) at the ortho position taken at 5 min of mixing (FIGS. 14a) and 30 min of mixing (FIG. 14b). The presence of peaks at m/z 266 and m/z 280 in the deconvoluted mass spectrum indicate the presence of phenol and o-cresol (1o), respectively.

Although the phenol and o-cresol in the indophenols have equal concentrations, at 5 minutes the signal for the cresol is nearly 25 times larger than that for phenol. However, after 30 minutes, the absolute signal for phenol increases by nearly a factor of 5, whereas that for o-cresol increases by less than 5%, which shows that under these buffer conditions, the reaction with o-cresol occurs much faster than that with phenol.

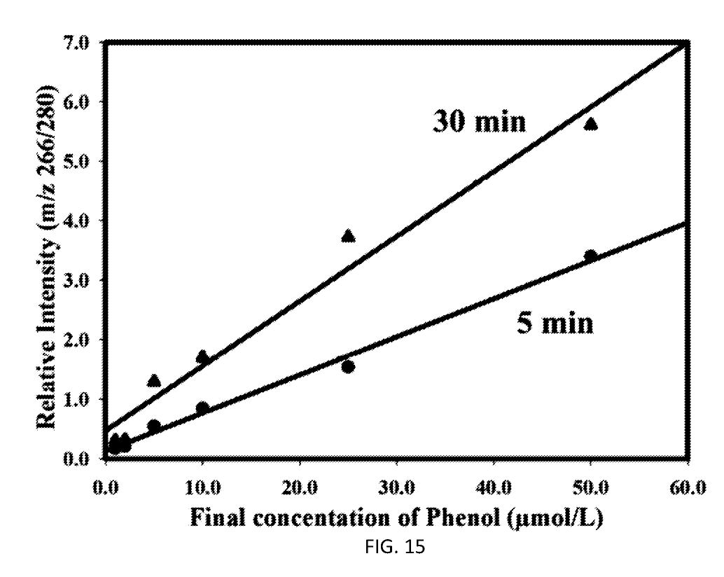

To use this method for the quantitative detection of phenol, a phenolic compound not present in the compound being tested can be used as an internal standard. Importantly, the compound under test (CUT) should be evaluated to determine what phenolic compounds exist in the CUT (based on the above described methods), then a phenolic compound not present in the CUT can be used as an internal standard based on an indophenol having an m/z separation of at least 5 as compared to the m/z of all other peaks found in the spectra. In one example o-cresol having a concentration of 3.0 μmol/L is used as an internal standard, although a host of other phonolic compounds are also possible. Calibration curves for the ratio I(m/z 266)/I(m/z 280) vs phenol concentration, for samples where the above-stated internal standard at 5 min and 30 min post-mix are shown in FIG. 15, which is a graph of intensity vs. concentration of phenols in μmol/L. This graph (FIG. 15) can then be used to quantify concentration of phenol compounds. Under the conditions used according to the present disclosure, the reaction of the Gibbs reagent with o-cresol is faster than that with phenol, such that the relative amount of the Gibbs product for phenol, m/z 266, compared to the Gibbs product for phenol, m/z 280, at longer reaction time is larger than that at short reaction time (based on a comparison of 30 minutes and 5 minutes reaction times). Nonetheless, the ratio of phenol Gibbs product to o-cresol Gibbs product is essentially linear (r² is about 0.99) over the range of phenol concentration from 1-50 μmol/L.

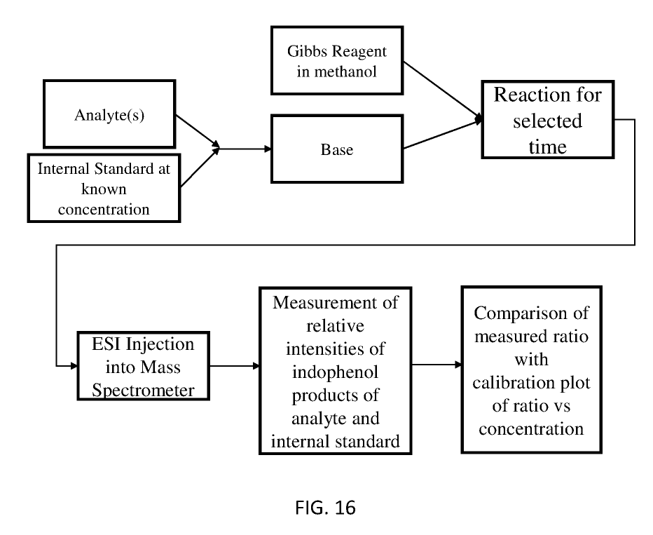

The quantification approach described above is shown in FIG. 16, which is a flowchart of the quantification aspect of the present disclosure. Initially the analyte is mixed with a buffer and mixed with the Gibbs reagent for a selected time (producing an indophenol) which is then provided to the mass spectrometer for analysis of what phenolic compounds are present. Once the phenolic compounds are identified, then a phenolic compound as an internal standard at a known concentration is chosen and the process repeated, this time with the internal standard generating a peak not seen in the previous run. Next, relative intensities of indophenol products of analyte and internal standard are obtained followed by a comparison of measured ratio with calibration plot of ratio vs concentration.

With the above description of the novel method, two compounds with unknown phenolic contents are now analyzed based on this method. Hickory, mesquite, pecan, and apple liquid smoke compounds are now described.

The mass spectrometric detection of Gibbs products can be used to analyze any sample that contains significant amounts of phenols. An example of this type of product is “liquid smoke,” a common additive used for flavoring and preservation, which includes wood smoke condensates. Phenols make up approximately ⅓ of commercially available liquid smoke, with guaiacol (2o), pyrocatechol (3o) and syringol (8) (shown below) are among the most important components. As an illustration of how this method can be used to analyze mixtures, four commercially available liquid smoke products, sold by The COLGIN COMPANIES (Dallas, Tex.), a “Natural Hickory”, “Natural Mesquite”, “pecan”, and “apple” are analyzed to determine the differences in phenolic composition.

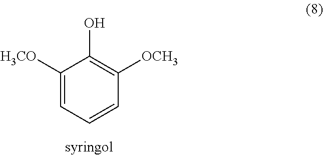 [SMILES: COc1cccc(OC)c1O]

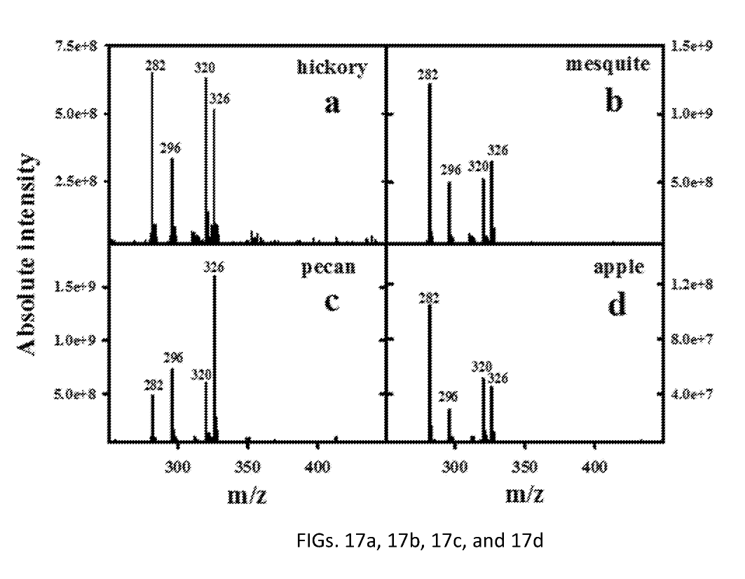

The deconvoluted mass spectra for hickory, mesquite, pecan, and apple flavors of liquid smoke upon reaction with the Gibbs reagent, taken 5 minutes after mixing, are shown in FIGS. 17a, 17b, 17c, and 17d which are deconvoluted spectra for hickory (17a), mesquite (17b), pecan (17c), and apple (17d). Once substituents are observed, then an internal standard having an m/z separation of at least 5 is chosen. In this case, an internal standard of 5,6,7,8-tetrahydro-1-napthol (THN) is used which produces an indophenol with a peak at m/z 320.

In FIG. 17a, significant peaks are at m/z 282, 296, 326 for Gibbs product of catechol, guaiacol, and syringol, respectively. As mentioned, the peak at m/z 320 is due to the Gibbs product of internal standard, THN. The spectra show that hickory contains more methoxy phenol than mesquite (17b), which is abundant with hydroxy phenol. Pecan flavored has mostly syringol (m/z 326) (FIG. 17c) and apple flavored has mostly catechol (m/z 282) (FIG. 17d). Interestingly, for this analysis 2 mL of the corresponding liquid smoke is taken for all flavored liquid smokes but apple, which is 8 mL to get significant signal of the Gibbs product.

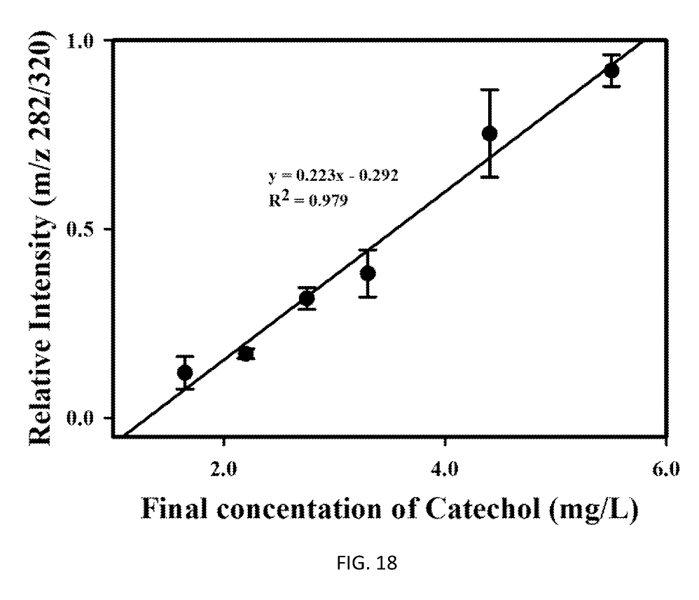

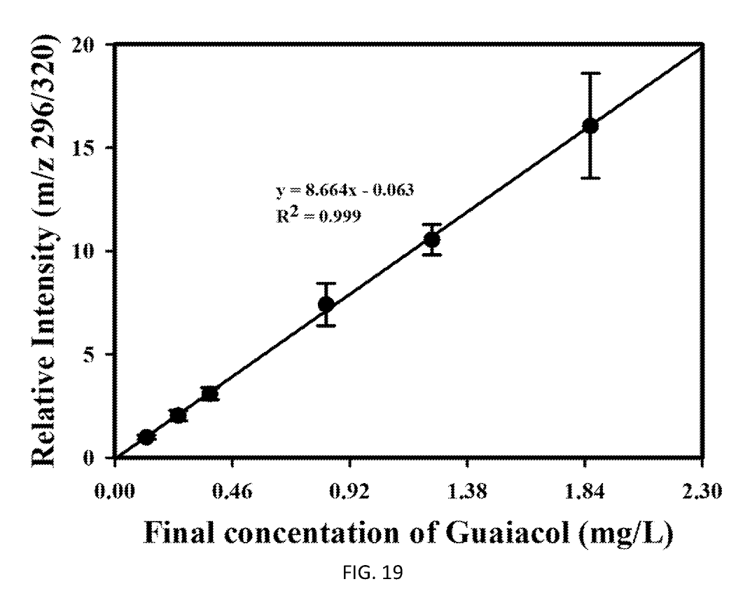

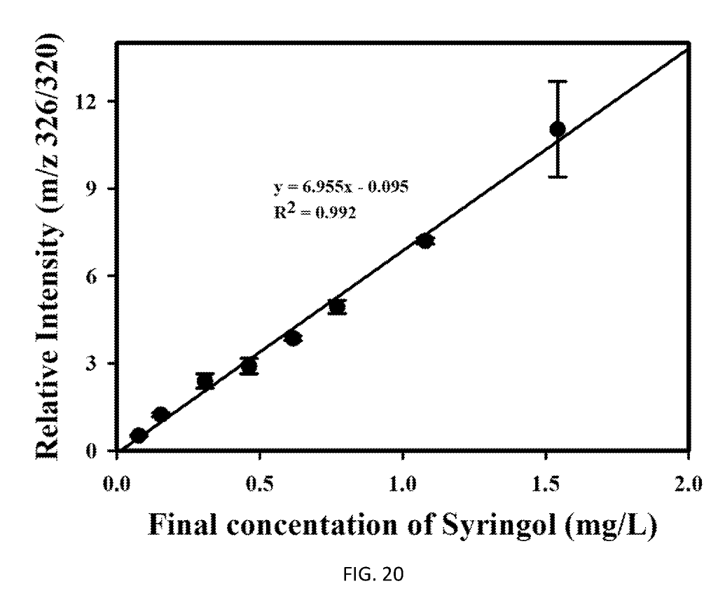

In each case of forming calibration curves, the intensity ratio of the corresponding compound and that of the internal standard, i.e., 5,6,7,8-tetrahydro-1-napthol (THN), is provided against the concentration of the corresponding compound after five minutes reaction time. FIGS. 18, 19, and 20 show the calibration curves by plotting I(m/z282)/I(m/z320) vs. catechol concentration (FIG. 18), I(m/z 296))/I(m/z320) vs. guaiacol concentration (FIG. 19), I(m/z 326)/I(m/z320) vs. syringol concentration (FIG. 20), respectively, where 5,6,7,8-tetrahydro-1-napthol (m/z 320) Gibbs products is used as the internal standard in each. The 5,6,7,8-tetrahydro-1-napthol is present at a concentration of 0.20 mg/L. The ratio of catechol Gibbs product to the internal standard Gibbs product is substantially linear (r²=0.993) over the range of catechol concentration from 1.65-5.50 mg/L. Similarly, in the other two calibration curves the ratios are substantially linear (r²=0.997 for guaiacol, and r²=0.998 for syringol) over the range of guaiacol concentration of 0.12-1.86 mg/L and over the concentration of syringol of 0.08-1.08 mg/L.

The concentrations (milligrams per liter and mmole per liter) of the phenolic compounds are presented in Table 1. The results are of an average of at least five set of experiments. The results show that the mostly plentiful compound in all four-different flavored liquid smoke is catechol and the least present amount is guaiacol. Catechol is present in mesquite flavored as high as 6.728±0.687 g/L (61.051±1.530 mmol/L). However, a low amount of catechol is contained in apple flavored liquid smoke (1.486±0.045 g/L, 13.488±0.409 mmol/L). Hickory flavored liquid smoke contains approximately half of catechol (3.444±0.141 g/L, 31.257±1.277 mmol/L) as compared to mesquite flavored, whereas pecan contains almost double of it (2.773±0.084 g/L, 25.167±0.759 mmol/L) than the apple flavored. The compound with the lowest amount contained is guaiacol, which is mostly present in pecan flavored (71.937±2.683 mg/L, 0.602±0.059 mmol/L) and least amount present in apple flavored liquid smoke (3.651±0.174 mg/L, 0.029±0.001 mmol/L). Interestingly, presence of guaiacol in mesquite flavored liquid smoke (61.672±3.553 mg/L, 0.497±0.029 mmol/L) is quite similar of pecan flavored and hickory flavored which contain around one-third guaiacol (23.029±2.654 mg/L, 0.186±0.021 mmol/L) of that in mesquite flavored. The third compound present in these liquid smokes is syringol. Pecan flavored contains the highest amount of it (209.420±8.963 mg/L, 1.351±0.086 mmol/L), while apple flavored liquid smoke contains lowest amount (20.904±0.642 mg/L, 0.132±0.004 mmol/L). Hickory (91.417±5.022 mg/L, 0.579±0.033 mml/L) and mesquite (94.779±5.508 mg/L, 0.601±0.036 mmol/L) flavored contain similar amount of syringol.

TABLE 1
Concentration of catechol, guaiacol, and syringol in mmol/L and mg/L in four different
flavored liquid smoke

| | Pecan | | Apple | | Hickory | | Mesquite | |
|---|---|---|---|---|---|---|---|---|
| Analyte | mg/L | mM | mg/L | mM | mg/L | mM | mg/L | mM |
| Catechol | 2772.680 | 25.167 | 1486.470 | 13.488 | 3444.196 | 31.257 | 6728.430 | 61.051 |
| | (83.677) | (0.759) | (45.127) | (0.409) | (140.810) | (1.277) | (168.696) | (1.530) |
| Guaiacol | 71.937 | 0.602 | 3.651 | 0.029 | 23.029 | 0.186 | 61.672 | 0.497 |
| | (2.683) | (0.059) | (0.174) | (0.001) | (2.654) | (0.021) | (3.553) | (0.029) |
| Syringol | 209.420 | 1.351 | 20.904 | 0.132 | 91.417 | 0.579 | 94.779 | 0.601 |
| | (8.963) | (0.086) | (0.642) | (0.004) | (5.022) | (0.033) | (5.508) | (0.036) |

In a detailed analysis of Code 10-Poly full-strength liquid smoke, Montazeri et al. found additional, less abundant products, including isomeric methoxymethylphenols and 3-methoxy-1,2-benzenediol. These appear to be present in the spectra in FIGS. 17a and 17b, at m/z 310 and 312, respectively. However, in both spectra, there is a product at m/z 311 that is similar in abundance to those at m/z 310 and 312. The odd m/z value would require a nitrogen containing product, and could, in principle, be attributed to an amino-methoxyphenol. Additional products are also observed at m/z 349, 351 and 420. The product at m/z 420 is important because, while a minor peak in the analysis at 5 min, it is the most abundant product at 30 min. From the isotope pattern and collision induced dissociation experiments, it is found that the product at m/z 420 contains chlorine. Since the reactions of hydroxy- and methoxyphenols with Gibbs reagents are fast, then the relative intensities of the peaks in FIGS. 17a and 17b reflect the relative concentrations of the phenolic components.

Those having ordinary skill in the art will recognize that numerous modifications can be made to the specific implementations described above. The implementations should not be limited to the particular limitations described. Other implementations may be possible.

1. A method of identifying phenolic compounds in a compound under test (CUT), comprising:
mixing the CUT with a buffer solution to generate a buffered compound;
mixing the buffered compound with a Gibbs reagent and allowing reaction for a predetermined amount of time to generate an indophenol;
inputting the indophenol to a mass spectrometer;
generating spectra of the indophenol; and
analyzing the spectra to determine presence of phenolic compounds in the indophenols.

2. The method of claim 1, wherein the buffered solution is basic.

3. The method of claim 2, wherein the buffered solution is potassium phosphate dibasic.

4. The method of claim 1, wherein a peak at m/z of 280 represents the indophenol of cresol (R═CH₃) at an ortho or meta positions, a peak at m/z of 296 represents the indophenol of hydroxyanisole (R═OCH₃) at the ortho or meta positions, a peak at m/z of 282 represents the indophenol of catechol or resorcinol (R═OH), a peak at m/z of 281 represents the indophenol of aminophenol (R═NH₂) at the ortho or meta positions, a peak at m/z of 316 represents the indophenol of 1-naphthol, and a peak at m/z of 320 represents the indophenol of tetrahydro-1-napthol.

5. The method of claim 1, wherein the Gibbs reagent is one of 2,6-dichloroquinone-4-chloroimide and 2,6-dibromoquinone-4-chloroimide.

6. The method of claim 5, further comprising deconvoluting the generated spectra and whereby the analyzing step is based on the deconvoluted step, where the spectral deconvolution removes peaks that are unrelated to double chlorine or bromine ions present in the indophenols.

7. The method of claim 1, wherein the mass spectrometer is an electrospray ionization mass spectrometer.

8. A method of identifying isomeric phenolic compounds in a compound under test (CUT), comprising:
mixing the CUT with a buffer solution to generate a buffered compound;
mixing the buffered compound with a Gibbs reagent and allowing reaction for a predetermined amount of time to generate an indophenol;
inputting the indophenol to a mass spectrometer;
generating spectra of the indophenol;
disassociating ions utilizing a collision induced disassociation (CID) stage; and
analyzing the spectra to determine i) presence of phenolic compounds in the indophenols, and ii) identify isomers of the phenolic compounds.

9. The method of claim 8, wherein the buffered solution is basic.

10. The method of claim 9, wherein the buffered solution is potassium phosphate dibasic.

11. The method of claim 8, wherein a peak at m/z of 280 represents the indophenol of cresol (R═CH₃) at an ortho or meta positions, a peak at m/z of 296 represents the indophenol of hydroxyanisole (R═OCH₃) at the ortho or meta positions, a peak at m/z of 282 represents the indophenol of catechol or resorcinol (R═OH), a peak at m/z of 281 represents the indophenol of aminophenol (R═NH₂) at the ortho or meta positions, a peak at m/z of 316 represents the indophenol of 1-naphthol, and a peak at m/z of 320 represents the indophenol of tetrahydro-1-napthol.

12. The method of claim 8, wherein the Gibbs reagent is one of 2,6-dichloroquinone-4-chloroimide and 2,6-dibromoquinone-4-chloroimide.

13. The method of claim 12, further comprising deconvoluting the generated spectra and whereby the analyzing step is based on the deconvoluted step, where the spectral deconvolution removes peaks that unrelated to double chlorine or bromine ions present in the indophenols.

14. The method of claim 8, wherein for an indophenol of cresol (R═CH₃) at an ortho position, the CID spectrum represents peaks at m/z of 198, 216, 244, 265, and 280; for an indophenol of cresol (R═CH₃) at a meta position, the CID spectrum represents peaks at m/z of 180, 198, 208, 216, 243, 244, 252, 264, and 280; for an indophenol of catechol (R═OH), the CID spectrum represents peaks at m/z of 218, 246, 254, and 282; for an indophenol of resorcinol (R═OH), the CID spectrum represents peaks at m/z of 109, 161, 210, 246, 254, 265, and 282; for an indophenol of aminophenol (R═NH₂) at the ortho position, the CID spectrum represents peaks at m/z of 181, 199, 209, 235, 245, 265, and 281; for an indophenol of aminophenol (R═NH₂) at the meta position, the CID spectrum represents peaks at m/z of 181, 209, 245, 265, and 281; for an indophenol of guaiacol, the CID spectrum represents peaks at m/z of 281, and 296; and for an indophenol of meta-hydroxyanisole (R═OCH₃), the CID spectrum represents peaks at m/z of 122, 253, 281, and 296.

15. A method of quantifying phenolic concentration of compounds in a compound under test (CUT), comprising:
mixing the CUT with a buffer solution to generate a buffered compound;
mixing the buffered compound with a Gibbs reagent and allowing reaction for a first predetermined amount of time to generate a first set of one or more indophenols;
inputting the first set of one or more indophenols to a mass spectrometer;
generating spectra of the first set of one or more indophenols;
identifying a first set of one or more peaks associated with the first set of one or more indophenols;
identifying a phenolic compound to be used as an internal standard with a peak for a corresponding indophenol of the internal standard at an m/z having a separation from the first set of one or more peaks of at least 5;
mixing the first set of one or more indophenols with the internal standard having a predetermined concentration for a second predetermined amount of time to generate a second set of one or more indophenols;
inputting the second set of one or more indophenols to the mass spectrometer;
generating spectra of the second set of one or more indophenols;
generating calibration curves for each of the first set of one or more indophenols, wherein the calibration curve represents a ratio of intensity of each of the first set of one or more indophenols to intensity of the internal standard vs. concentration of each of the first set of one or more indophenols; and
obtaining the concentration of each of the first set of indophenols.

16. The method of claim 15, wherein the buffered solution is basic.

17. The method of claim 16, wherein the buffered solution is potassium phosphate dibasic.

18. The method of claim 15, wherein a peak at m/z of 280 represents the indophenol of cresol (R═CH₃) at an ortho or meta positions, a peak at m/z of 296 represents the indophenol of hydroxyanisole (R═OCH₃) at the ortho or meta positions, a peak at m/z of 282 represents the indophenol of catechol or resorcinol (R═OH), a peak at m/z of 281 represents the indophenol of aminophenol (R═NH₂) at the ortho or meta positions, a peak at m/z of 316 represents the indophenol of 1-naphthol, and a peak at m/z of 320 represents the indophenol of tetrahydro-1-napthol.

19. The method of claim 15, wherein the Gibbs reagent is one of 2,6-dichloroquinone-4-chloroimide and 2,6-dibromoquinone-4-chloroimide.

20. The method of claim 15, further comprising deconvoluting the generated spectra and whereby the analyzing step is based on the deconvoluted step, where the spectral deconvolution removes peaks that are unrelated to double chlorine or bromine ions present in the indophenols.
